(19) United States
(12) Patent Application Publication
(10) Pub. No.: US 2023/0234932 A1
(43) Pub. Date: Jul. 27, 2023

(54) SYNTHESIS AND APPLICATION OF DILTIAZEM HYDROCHLORIDE

(71) Applicant: Hainan Jin Rui Pharmaceutical Co., Ltd., Haikou City, Hainan (CN)

(72) Inventors: Shuhua PAN, Haikou City, Hainan (CN); Jinyu WANG, Haikou City, Hainan (CN); Shouchun WANG, Haikou City, Hainan (CN)

(21) Appl. No.: 17/598,611

(22) PCT Filed: Sep. 14, 2021
(86) PCT No.: PCT/CN2021/118177

§ 371 Date: Sep. 27, 2021

(30) Foreign Application Priority Data
Jun. 8, 2021 (CN) 202110638156.2

(51) Int. Cl. C07D 281/10

(52) U.S. Cl. CPC C07D 281/10

(57) ABSTRACT

The invention belongs to the field of drug synthesis, the invention discloses a synthetic method and application of diltiazem hydrochloride. The synthesis method is through the condensation ring closing and esterification of cis-(2S,3S) compound 1 and compound 2, then add hydrochloric acid to form salt, get the diltiazem hydrochloride. The invention has the following advantages, shortens the synthesis steps, improves the reaction yield, reduces the industrial production cost, and saves the time cost of the synthesis. The method is used for the synthesis of diltiazem hydrochloride, and the synthesized diltiazem hydrochloride is used to prepare diltiazem hydrochloride for injection.

### FIELD OF THE INVENTION

The invention belongs to the field of drug synthesis, which relates to a synthetic method and application of cardiovascular drugs, specifically, it is a synthetic method and application of diltiazem hydrochloride.

### BACKGROUND OF THE INVENTION

Diltizaem, a cardiovascular drug, is a benzothiadiazepine calcium channel blocker, Antagonists have obvious effect on dilating coronary vessels, clinically, it is mostly used for the treatment of cardiovascular diseases such as arrhythmia, angina pectoris and hypertension. The chemical name of diltiazem hydrochloride is cis-(2S,3S)-(+)-5-[(2-dimethylamino) ethyl]-2-(4-methoxyphenyl)-3-acetoxy-2,3-dihydro-1,5-benzothiadiazepine-4 (5 hydrogen)-one, the structural formula is:

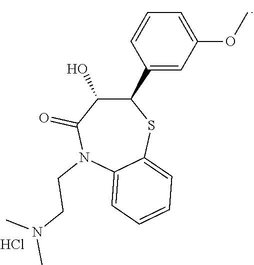

Diltiazem hydrochloride has two chiral centers, only (2S,3S)-isomers have pharmacological activity. The synthesis of diltiazem hydrochloride is based on chemical resolution, asymmetric synthesis and synthesis using biological enzymatic resolution techniques: Chemical resolution is currently the most applied method for the synthesis of diltiazem, many literatures have reported its chemical synthesis route and corresponding resolution methods, The synthetic routes currently used in large-scale production all use anisol as the raw material to obtain the final product through multi-step reactions. The corresponding resolution reagents for diltiazem hydrochloride have been comprehensively studied. The earliest report was the use of cinchonidine to resolve diltiazem. The disadvantage of chemical separation is that its resources are lost and wasteful, and the cost of the entire route is high. Then use L-lysine or 1-ephedrine to resolve intermediates, Lysine has higher optical purity and higher yield than ephedrine resolution, In addition, 1-lysine is cheaper, so 1-lysine is used. Acid resolution has been widely used in industry, but the synthetic route is still lengthy, and the theoretical yield of the reaction is up to 50%, which will waste a lot of resources.

The asymmetric synthesis method uses a catalyst to carry out asymmetric dihydroxylation or epoxidation reaction, but the catalyst used makes the synthesis cost high, which is not conducive to large-scale industrial production.

Biological enzymatic hydrolytic resolution is a method for the resolution of diltiazem hydrochloride by lipase catalysis, lipases are a class of commonly used biocatalysts, intermediates with EE>99% were eventually obtained, and the synthetic route of diltiazem can be reduced from the original 9-step reaction commonly used in industrialization to 5 steps. But biological enzyme resolution also has many problems, for example, the utilization rate of the currently available split products is still uncertain. These are problems that need to be solved urgently in reality.

The synthesis process of diltiazem hydrochloride has received much attention in recent years. A process route with high yield, low cost, mild reaction conditions, simple operation, green, and industrialized large-scale production was developed to meet the needs of the society for this type of medicine, and it has received general attention from the organic chemistry community.

### DETAILED DESCRIPTION OF THE INVENTION

In order to solve the above shortcomings in the prior part, the invention aims to provide a synthesis method of diltiazem hydrochloride, in order to achieve the purpose of increasing the reaction yield, reducing the reaction steps, and reducing the synthesis cost.

In order to achieve the above purpose, the technical scheme adopted by the invention is as follows:

A synthetic method of diltiazem hydrochloride, wherein the synthetic method is:

Step 1)cis-(2S,3S) compound (1) and compound (2) were mixed into an organic solvent, add cuprous iodide and an organic base to carry out a condensation ring-closing reaction, after the reaction, compound (3) was obtained;

Step 2) the compound (3) is esterified with acetic acid or acetic anhydride in 1,4-dioxane. After the reaction, adjust the pH to alkaline with sodium carbonate, and add hydrochloric acid to obtain diltiazem hydrochloride. The reaction formula is:

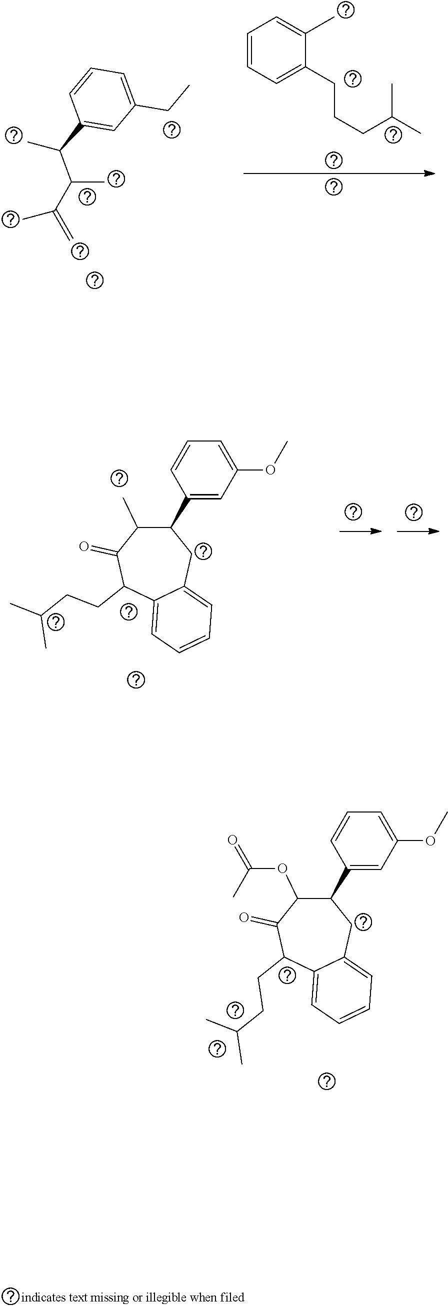

As a limitation of the invention, wherein the organic solvent is tetrahydrofuran, N,N-dimethylformamide or N-methylpyrrolidone; The organic base is triethylamine, N,N-Diisopropyl ethylamine or sodium ethanol,

As another limitation of the invention, the reaction temperature of the condensation ring closing reaction is 85^{˜}120° C. and the reaction time is 1^{˜}5 h;

As a third limitation of the invention, the reaction temperature of the esterification reaction is 100^{˜}120° C. and the reaction time is 2^{˜}4 h;

The invention also provides an application of the synthetic method of diltiazem hydrochloride, which is used for synthesise diltiazem hydrochloride; The synthesized diltiazem hydrochloride is used for preparing diltiazem hydrochloride for injection.

The benefits are:

(1) The synthetic process for diltiazem hydrochloride as provided in this invention directly uses compound 1 which is cis-(2S,3S) after resolution, during the reaction, the resolution process to the intermediate is reduced, improved reaction yield, eliminates the use of split reagents, reduced industrial synthetic costs.

(2) Diltiazem hydrochloride can be synthesized in only 2 steps, and the reaction time is also greatly shortened during the synthesis process, Saving a lot of time and cost

(3) Toxicity of reagents is low, the process of participating in the reaction is also relatively mild, produces less three wastes.

### BRIEF DESCRIPTION OF THE DRAWINGS

The invention is described in more detail below in combination with the accompanying drawings and specific examples.

FIG. 1 is an HPLC diagram of diltiazem hydrochloride A1 synthesized in example 1 of the present invention.

### EXAMPLES

A preferred example of the present invention is described below in combination with the accompanying drawings. It should be understood that the preferred examples described herein are only used to illustrate and understand the invention and are not used to limit the invention.

Example 1: a synthetic method of diltiazem hydrochloride A1

This example provides a synthetic method of diltiazem hydrochloride A1, which is as follows:

1) Dissolved 24.6 g cis-(2S,3S) compound 1 and 29 g compound 2 in 150 ml THF, add 15 g tea, stir evenly, add 0.95 g cuprous iodide, stir continuously and heat up to 90° C., keep stirring for 4 h, after 4 h reaction, do TLC (thin layer chromatography) to detect the reaction, take a small amount of the reaction solution diluted with ethyl acetate (EA), dip it in a capillary tube and spot it on the silica gel plate as a sample spot; and dilute a small amount of compound 2 and compound 1 as the reference point, use the capillary point and the sample point at the same level, put the silicone plate into the pre-configured mixed solution of petroleum ether:ethyl acetate=10:1, and the liquid level of the solution does not exceed the level of the sample point and the reference point, stand for 3 min, take it out and observe it under the UV lamp at 254 nm, there is a point with less polarity than compound 1 and compound 2 in the sample point, and there is no point with compound 1 and compound 2 in the sample point, that is, the reaction is complete.

After the reaction, reduce the reaction solution to room temperature, add 300 ml ethyl acetate and 100 ml water, stir evenly, stand for layering, collect the organic phase and decolorize it by simple column chromatography (eluent: EA), collect and concentrate the eluent to dry, so as to obtain 33.1 g compound 3 with a yield of 89%.

2) Dissolve 33.1 g of compound 3 obtained in the above steps in 180 ml dioxane, add 15.4 ml of acetic acid, continue to stir and raise the temperature to 100° C., keep the reaction for 2 h, take a small amount of sample for dilution after 2 h, and then inject it into the liquid chromatograph for normal conditions Analysis (acetonitrile:methanol=20:1), the absorption peak of compound 3 disappeared, and an absorption peak with a polarity smaller than that of compound 3 appeared, and the molecular weight of the absorption peak showed 415 (M+), that is, the end of reaction.

After the reaction, reduce the reaction solution to room temperature, concentrate it to dry, add 50 ml dichloromethane (DCM) and stirring, add saturated sodium carbonate aqueous solution, continue stirring until the system mixture is clear, stand for layering, retain the organic phase, add 100 g activated carbon for decolorization, filter, concentrate the filtrate to dry, and then add 30 ml absolute ethanol for dissolution, Add 38% hydrochloric acid dropwise until a large amount of solid precipitates from the system, filtrate it, wash the filter cake with is 100 ml absolute ethanol for 3 times, dry and crush the filter cake to obtain 35.4 g diltiazem hydrochloride A1, with a yield of 96%, a total yield of 85.4% and a purity of 99.6%. Take a small amount of samples and measure the EE value by capillary electrophoresis, the EE=99.1%. The reaction formula is:

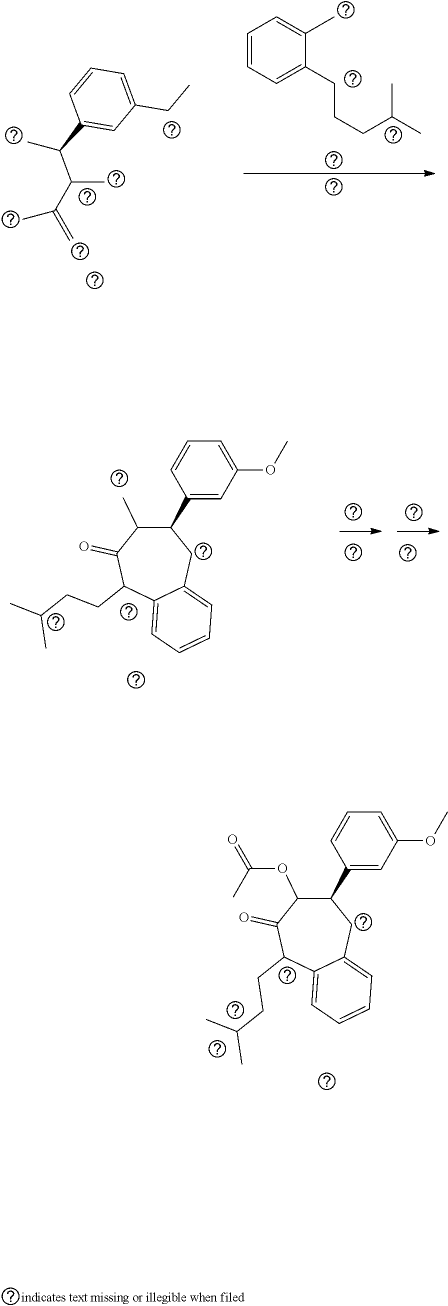

Take a small amount of diltiazem hydrochloride A1,

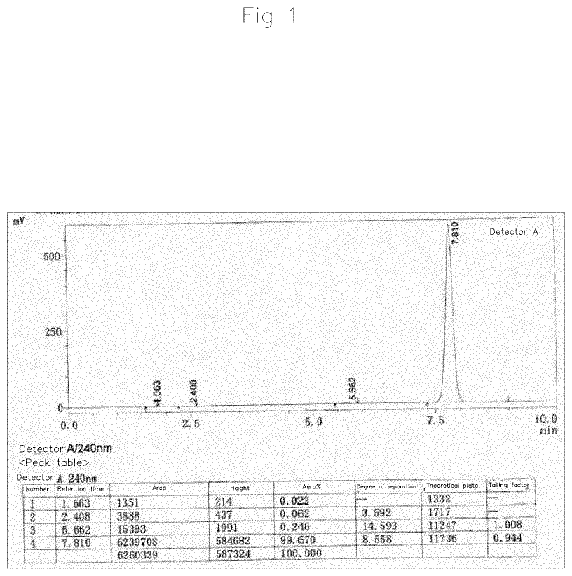

Take a small amount of diltiazem hydrochloride A1, dissolve it, and carry out HPLC detection according to the method described in general rule 0512 of Part IV of Chinese Pharmacopoeia. The obtained HPLC diagram is shown in FIG. 1.

Examples 2-6 is synthetic method of diltiazem hydrochloride a2-a6.

The synthetic methods of diltiazem hydrochloride A2^{˜}A6 provided in examples 2^{˜}6 are basically the same as those described in example 1, except that some process parameters are different. See Table 1 for specific process parameters.

TABLE 1
process parameters of diltiazem hydrochloride A2A6

| | | | examples | | | | |
|---|---|---|---|---|---|---|---|
| process parameters | | | 2 | 3 | 4 | 5 | 6 |
| 1) | Organic | THF | — | — | 100 | — | — |
| | solvent | DMF | 20 | — | — | 50 | — |
| | (ml) | DMSO | — | 50 | — | — | 80 |
| | Organic | TEA | — | — | 20.2 | — | — |
| | base | DIPEA | 19.4 | — | — | 25.8 | — |
| | (g) | EtO Na | — | 10.2 | — | — | 13.6 |
| | Condensation | Temp | 85 | 110 | 100 | 120 | 95 |
| | ring closing | (° C.) | | | | | |
| | reaction | Time (h) | 5 | 3 | 4 | 1 | 2 |
| | | Yield (%) | 88 | 90 | 90 | 91 | 89 |
| 2) | Acetic acid (ml) | | 7.5 | — | 5.6 | — | 10.1 |
| | Acetic anhydride (ml) | | — | 12.8 | — | 9.5 | — |
| | Esterification | Temp | 110 | 120 | 105 | 100 | 115 |
| | | (° C.) | | | | | |
| | | Time (h) | 3 | 2 | 3 | 4 | 2 |
| | | Yield (%) | 98 | 96 | 95 | 95 | 97 |
| Diltiazem | | Number | A2 | A3 | A4 | A5 | A6 |
| hydrochloride | | Purity | 99.4 | 99.2 | 99.7 | 99.7 | 99.8 |
| | | (%) | | | | | |
| | | Total yield | 85.4 | 88.2 | 86.3 | 87.1 | 85.8 |
| | | (%) | | | | | |
| | | ee (%) | 99.4 | 99.1 | 99.3 | 99.4 | 99.2 |

Other parameters are the same as those in example 1.

Comparative Example 1^{˜}4, the effect of temperature on diltiazem hydrochloride.

For the synthesis methods of diltiazem hydrochloride provided in Comparative Example 1^{˜}4, their synthesis methods are basically the same as those described in example 1, except that the reaction temperatures of each step are different. See Table 2 for specific reaction temperature parameters.

TABLE 2
Diltiazem hydrochloride reaction temperature parameter table
for each step.

| Comparative | | Temp | | yield |
|---|---|---|---|---|
| Example | step | (° C.) | Reaction results | (%) |
| 1 | 1) | 80 | The reaction was incomplete and | 55.9 |
| | | | both compound 1 and compound | |
| | | | 2 remained | |
| 2 | | 130 | Compound 3 racemic, ee = 0 | — |
| 3 | 2) | 95 | Compound 3 remained more and | 57.8 |
| | | | the reaction was not thorough | |
| 4 | | 130 | Diltiazem hydrochloride | — |
| | | | racemic, ee = 0 | |

It can be seen from the results that the reaction temperature has an obvious effect on the synthesis of diltiazem hydrochloride, and too high temperature will affect the EE value of the final product diltiazem hydro chloride, then affecting the drug activity; When the temperature is low, the reaction will be incomplete, which will affect the yield of the reaction.

Application example, Preparation of diltiazem hydrochloride for injection.

This application example provides a preparation method of diltiazem hydrochloride for injection. The raw material used is diltiazem hydrochloride in example 3 (purity 98%, EE: 99.1%), and the preparation method is as follows:

S1 material preparation, weigh 10 g diltiazem hydrochloride, 40 g mannitol and 5 g poloxamer for standby;

S2 solution preparation: prepare injection water of 80% of total solution, place it in the solution preparation tank, start stirring, add the prescribed amount of diltiazem hydrochloride raw material, mannitol and poloxamer while stirring, continue stirring until it is completely dissolved and the solution became clear, and supplement injection water to the full amount; The temperature of water for injection is 24° C., and the temperature of drug solution is maintained within 24° C.; Preferably, diltiazem hydrochloride raw material, mannitol and poloxamer are added into the liquid preparation tank at a uniform speed within 60 min;

S3 adjust the pH value, add sodium hydroxide solution to the solution preparation tank, and adjust the pH value of the solution to 5.6;

S4 filtration, the drug solution is received in a stainless steel barrel and weighed after intermediate detection; After the intermediate is qualified, the drug solution is transferred to the filter press tank and passed through 0.22 μM primary sterilization and filtration to the liquid medicine storage tank at the liquid medicine receiving place in the filling room; Move the liquid medicine storage tank under the class A laminar flow hood of the filling machine for 0.22 μM. sterilize and filter the secondary terminal into the liquid medicine barrel, adjust the filling volume according to the filling production instruction card, fill it into the injection bottle, half press the plug and place it on the shelf in the freeze-drying cabinet;

S5 freeze drying, pre-freezing first, drying again, and finally secondary drying; Among them, pre-freezing means that the shelf temperature is controlled at 120° C. and the shelf temperature is reduced to −50° C. for more than 90 minutes after the product is put into the box. When the product temperature reaches −35° C., it is kept warm for 2.5 hours; The primary drying is to reduce the condenser temperature to −50° C., vacuum the system, reduce the vacuum degree to 10 Pa, raise the temperature of the shelf to −6° C. in 90 minutes, and continue the heat preservation for 3 hours after the ice crystal of the product disappears completely; The second drying is that the shelf rises to 26° C. within 60 minutes, and the product temperature rises to 20° C. for 4.5 hours; The total time of freeze-drying step is about 14 hours;

S6. After passing the inspection, the freeze-dried samples shall be subject to capping and light inspection, and then the capping and light inspection qualified samples shall be labeled and packed. After passing the inspection, the finished products shall be warehoused.

Stability test of diltiazem hydrochloride for injection According to the stability test conditions described in general rule 90 01 of Part IV of Chinese Pharmacopoeia (including high temperature test, high humidity test, strong light irradiation test, accelerated test and long-term test), The diltiazem hydrochloride for injection prepared in the above application example and the diltiazem hydrochloride for injection sold on the market (specification of Zhejiang Asia Pacific Pharmaceutical Co., Ltd.:10 mg/piece) are used as experimental examples and comparative tests for stability respectively. The specific experimental data are shown in Table 3.

TABLE 3
stability test data of diltiazem hydrochloride for injection

| | | | | Purity* |
|---|---|---|---|---|
| Item | condition | object | character | % |
| High | 40° C. | example | White loose block; | 98.4 |
| temperature | 10 Day | | Odorless, slightly | |
| test | | | bitter taste | |
| | | Comparative | White loose block; | 98.0 |
| | | Example | Odorless, slightly | |
| | | | bitter taste | |
| High | 75% RH, | example | White loose block; | 99.2 |
| humidity | 10 Day | | Odorless, slightly | |
| test | | | bitter taste | |
| | | Comparative | White loose block; | 98.9 |
| | | Example | Odorless, slightly | |
| | | | bitter taste | |
| Strong light | 4500 Ix, | example | White loose block; | 97.8 |
| irradiation | 10 Day | | Odorless, slightly | |
| test | | | bitter taste | |
| | | Comparative | White loose block; | 96.3 |
| | | Example | Odorless, slightly | |
| | | | bitter taste | |
| Accelerated | 40° C. ± 2° C./ | example | White loose block; | 96.6 |
| test | 75% ± 5% | | Odorless, slightly | |
| | RH, 3 month | | bitter taste | |
| | | Comparative | White loose block; | 95.8 |
| | | Example | Odorless, slightly | |
| | | | bitter taste | |
| Long term | 25° C. ± 2° C./ | example | White loose block; | 95.2 |
| test | 60% ± 10% | | Odorless, slightly | |
| | RH, 24 month | | bitter taste | |
| | | Comparative | White loose block; | 94.1 |
| | | Example | Odorless, slightly | |
| | | | bitter taste | |

The purity* mentioned in the table is the purity of diltiazem hydrochloride for injection measured after the test.

It can be seen from the data in the table that the synthesis method provided by the invention not only has high yield, purity and EE value, but also The stability of diltiazem hydrochloride for injection prepared by it is better than that of the commercially available diltiazem hydrochloride for injection under the same storage environment. There is no change in properties, and the purity declines relatively slowly. Even after long-term tests, the purity is still at more than 95%, which meets the standards for injectable drugs in Chinese Pharmacopoeia.

It should be noted that the above descriptions are only preferred embodiments of the present invention and are not intended to limit the present invention. Although the invention is described in detail with reference to the above examples, those skilled in the art can still modify the technical solutions recorded in the above examples or replace some of the technical features equally. Any modification, equivalent replacement, improvement, etc. made within the spirit and principles of the invention shall be included in the protection scope of the invention.

1. A synthetic method of diltiazem hydrochloride, wherein the synthetic method is:
Step 1) cis-(2S,3S) compound (1) and compound (2) were mixed into an organic solvent, add cuprous iodide and an organic base to carry out a condensation ring-closing reaction, after the reaction, compound (3) was obtained;
Step 2) the compound (3) is esterified with acetic acid or acetic anhydride in 1,4-dioxane. After the reaction, adjust the pH to alkaline with sodium carbonate, and add hydrochloric acid to obtain diltiazem hydrochloride. The reaction formula is:

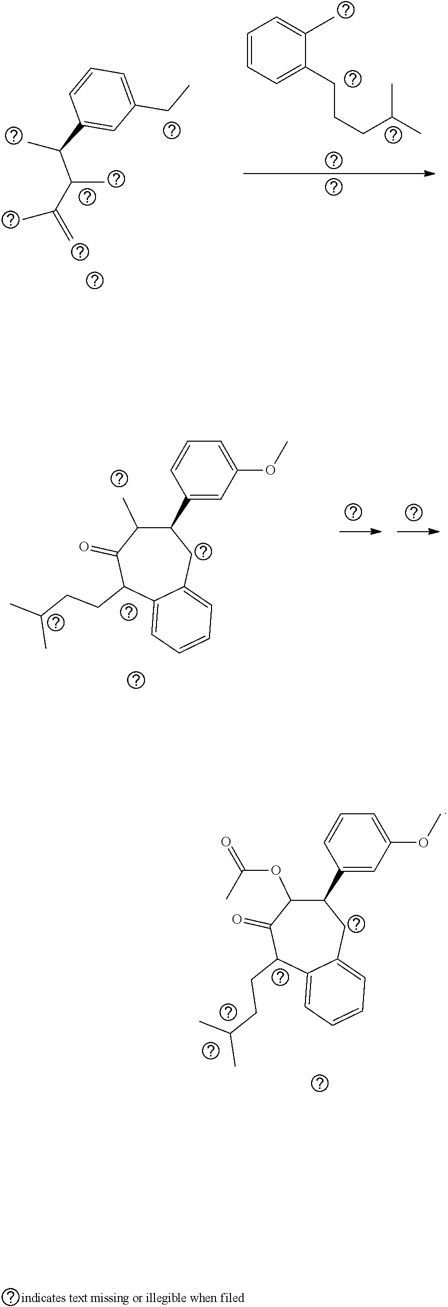

2. The method of claim 1, wherein the organic solvent is tetrahydrofuran, N,N-dimethylformamide or N-methylpyrrolidone; The organic base is trimethylamine, N,N-Diisopropyl ethylamine or sodium ethanol.

3. The method of claim 1, wherein the reaction temperature of the condensation ring closing reaction is 85˜120° C. and the reaction time is 1˜5 h.

4. The method of claim 1, wherein the reaction temperature of the esterification reaction is 100˜120° C. and the reaction time is 2˜4 h.

5. According to claim 1, an application of the method for synthesizing diltiazem hydrochloride, characterized in that the synthesis method is used to synthesize diltiazem hydrochloride; The synthesized diltiazem hydrochloride is used for preparing diltiazem hydrochloride for injection.

6. According to claim 2, an application of the method for synthesizing diltiazem hydrochloride, characterized in that the synthesis method is used to synthesize diltiazem hydrochloride; The synthesized diltiazem hydrochloride is used for preparing diltiazem hydrochloride for injection.

7. According to claim 3, an application of the method for synthesizing diltiazem hydrochloride, characterized in that the synthesis method is used to synthesize diltiazem hydrochloride; The synthesized diltiazem hydrochloride is used for preparing diltiazem hydrochloride for injection.

8. According to claim 4, an application of the method for synthesizing diltiazem hydrochloride, characterized in that the synthesis method is used to synthesize diltiazem hydrochloride; The synthesized diltiazem hydrochloride is used for preparing diltiazem hydrochloride for injection.
